(19) United States
(12) Patent Application Publication
(10) Pub. No.: US 2019/0021996 A1
(43) Pub. Date: Jan. 24, 2019

(54) Therapeutic Preparations and Articles

(71) Applicant: Gregory A. Rivera, Fort Lee, NJ (US)

(72) Inventor: Gregory A. Rivera, Fort Lee, NJ (US)

(21) Appl. No.: 16/069,470

(22) PCT Filed: Jan. 13, 2017
(86) PCT No.: PCT/US17/13488

§ 371 Date: Jul. 11, 2018

Related U.S. Application Data
(60) Provisional application No. 62/278,506, filed on Jan. 14, 2016.

(51) Int. Cl. A61K 9/06; A61K 9/10; A61K 33/32; A61K 47/10; A61K 9/70; A61Q 19/00; A61Q 19/08; A61K 8/02; A61K 8/04; A61K 8/34; A61K 8/26; A61K 9/51

(52) U.S. Cl. CPC A61K 9/06; A61K 2800/413; A61K 33/32; A61K 47/10; A61K 9/70; A61K 9/7053; A61Q 19/007; A61Q 19/08; A61K 8/0208; A61K 8/044; A61K 8/042; A61K 8/345; A61K 8/26; A61K 9/51; A61K 9/10

(57) ABSTRACT

A therapeutic preparation and article treated with tourmaline nano-particles and a method of manufacturing the same. The therapeutic preparation comprises a gel and tourmaline nano-particles held in suspension in said gel. The therapeutic article comprises a fabric and tourmaline particles attached to said fabric. In one embodiment, an adhesive containing tourmaline is applied to non-treated fabric to form the article. In another embodiment, the tourmaline is integral to the fabric. A method of manufacturing the therapeutic article comprises the steps of submerging a fabric in an aqueous solution comprising tourmaline nano-particles and drying said fabric.

### FIELD

The present disclosure relates to the field of therapeutic materials, and in particular, gels, adhesives, bandages and apparel treated with tourmaline.

### BACKGROUND

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Often times an individual is prevented from living a healthy life because of ailments to the body, and in particular muscle and bone damage. Therefore, medical and physical therapy is important to recovery and health. This is also a concern for athletes who endure constant wear and tear of muscles and bones.

Currently, certain products that provide temporary relief and therapeutic treatment to injured and/or ailing parts of the body exist. For example, electric heat pads can be used to provide temporary relief. However, electric heat pads are costly and are not portable. As another example, ultrasound is used to treat injured muscles and bones but this can only be done in a stationary setting. Furthermore, ultrasound treatment often requires the patient to have health insurance and even with health insurance the patient may incur costs out of pocket. In yet another example, heat patches are sold for temporary treatment to specific parts of the body. Heat patches, however, are not only expensive but are not versatile due to the predetermined shapes and sizes they are sold in, and are generally ineffective because they do not provide sufficient heat and because heat is generated only for a short period of time.

Therefore, broadly, an inexpensive and convenient means to treat targeted parts of the body with definite therapeutic and healing effects is needed.

Tourmaline is a crystalline mineral composed of several elements including, but not limited to, aluminum, iron, magnesium, sodium, lithium, and potassium. The word tourmaline takes it origins from the Singhalese phrase “tura mali,” meaning “stone mixed with vibrant colors.” In the gem form, tourmaline could be transparent or any translucent shade of red, pink, purple, yellow, green, blue, brown or amber, or any combination thereof. However, black tourmaline is believed by holistic practitioners to emit the most powerful healing energy of all tourmaline gems. Tourmaline has historically been revered as a “magic” stone capable of protecting whoever wore it.

Tourmaline is known to generate a weak electric current, producing far infrared rays and converting moisture in the air to negative ions. Unlike mechanically produced negative ions, those generated by tourmaline are completely natural and safe and have no negative side effects. In fact, many of the beneficial effects or tourmaline are thought to be brought about by the negative ions it emits.

The exothermic ionic energy emitted by tourmaline has many potential applications. Tourmaline is known to have therapeutic effects on the human body. For example, tourmaline is widely used as a semi-precious gemstone to provide beneficial health effects through its unique electric properties.

Specifically, tourmaline gemstones are known to aid in relieving stress, increase mental alertness, improve circulation and strengthen the immune system. It is also thought to purify the blood, stimulate the autonomic nervous system, calm overexcited sympathetic nerves, and promote sound sleep. Worn on the body, tourmaline is thought to prevent cholesterol coagulation and build-up in blood vessels and increase blood flow. It is also a powerful agent for decreasing swelling, killing bacteria, and reducing toxin-related ailments as cells throughout the body are invigorated and immune function is enhanced by tourmaline.

Another known application of tourmaline, in its powder form, are in cosmetics applications. Tourmaline is believed to vitalize the skin, making it appear more radiant and youthful. When tourmaline crystals are warm as they are rubbed onto skin, they become positively charged on one end and negatively charged on the other. Because of this unique feature, tourmaline has been incorporated into moisturizers, exfoliates, and anti-agers to increase the absorption of nutrients into the skin. According to one anti-aging product, tourmaline extract triggers a warming effect to increase the production of new collagen and the contraction of fibers, thus restoring the springiness of skin.

Although the therapeutic effects of tourmaline stones and possible benefits to skin are known, the use of tourmaline on targeted parts of the body for medical and physical therapy have not been developed.

As mentioned above, an inexpensive and convenient means to treat targeted parts of the body with definite therapeutic and healing effects is needed. Specifically, a need exists for the use of tourmaline for medical and physical therapy.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

The present invention provides an inexpensive and convenient means to treat targeted parts of the body with therapeutic and healing effects. Specifically, the present invention is for a portable therapeutic article treated with tourmaline so that specific body parts could be targeted for treatment. More specifically, the present invention is for tourmaline gel, tourmaline treated athletic tape and bandages, and tourmaline treated adhesives for shirts, pants, socks, braces, bands and bandages. The present invention also includes apparel manufactured with tourmaline treated fabric. The present invention is also related to the method of manufacturing the same.

The tourmaline products of the present invention utilize tourmaline nano-particles. These nano-particles could be in powder form or liquid form.

The tourmaline gel of the present invention comprises tourmaline nano-particles which are mixed with a binding agent. In gel form, for example, the tourmaline gel of the present invention may be used to reduce the energy requirements for ultrasounds. The tourmaline gel could also be used to treat muscle and joint aches, as well as cuts and bruises.

Thus, in one aspect, the present invention provides a therapeutic preparation comprising a binding agent and tourmaline particles held in suspension in said binding agent. The preparation is selected from a group comprising a gel, a cream, an ointment and a lotion. The binding agent comprises propylene glycol and the tourmaline particles have a diameter greater than 100 nanometers. The tourmaline particles are selected from a group comprising black tourmaline, white tourmaline and gray tourmaline. A concentration of the tourmaline particles is about 20% of an overall volume of the therapeutic preparation.

The tourmaline adhesive tape of the present invention comprises: (i) tourmaline nano-particles; (ii) adhesive; (iii) breathable and flexible base fabric; and (iv) release paper. The tourmaline adhesive tape of the present invention could be used to apply to specific parts of the body to aid in targeted healing and/or temporary pain relief.

In one embodiment, the fabric is treated with tourmaline and non-treated adhesive is applied to the treated fabric. In this embodiment, the method of manufacturing the tourmaline adhesive tape of the present invention comprises: (i) creating a solution by combining polyacrylic acid derivative, tourmaline powder, and water; (ii) applying the flexible base fabric to said solution; (iii) heating the said fabric; (iv) coating the fabric with adhesive; and (v) applying release paper. Alternatively, the solution could be a heavy water solution comprising deuterium oxide.

This method may require special equipment that can operate in the presence of tourmaline nano-particles which can leave residue that can potentially clog equipment, requiring additional maintenance, or cause premature wear and tear to the equipment.

In another embodiment, the adhesive is treated with tourmaline and applied to non-treated fabric. In this embodiment, the method of manufacturing the tourmaline adhesive tape of the present invention comprises: (i) mixing an adhesive with tourmaline nano-particles; (ii) applying the adhesive to the flexible base fabric; and (iii) applying release paper to the adhesive coating. Examples of the adhesive include polyurethane polymer and acrylic polymer.

To create a traditional athletic tape, the tourmaline adhesive tape would be conveyed through a rolling device to be rolled, sheared, wrapped, and boxed. To create a bandage, gauze can be attached to the fabric or a gauze with tourmaline can be conveyed to fabric with adhesive.

Thus, in another aspect, the present invention provides a therapeutic article comprising: a fabric and tourmaline particles attached to said fabric. The tourmaline particles are ionized into the fabric to about 500 ions per cubic centimeter. In one embodiment, an adhesive is applied to the fabric, wherein the tourmaline particles are included in the adhesive. The adhesive comprises N-Vinylpyrrolidone polymer and wherein a concentration of the tourmaline particles is about 20% of an overall volume of the adhesive. The adhesive is applied to the fabric by a method selected from a group comprising spraying, soaking, blotting and foaming. The fabric is capable of being stretched to at least 150% of its original length.

In another embodiment, the fabric is directly treated with tourmaline. Thus, in yet another aspect, the present invention provides a method of manufacturing a therapeutic article comprising the steps of: submerging a fabric in an aqueous solution comprising tourmaline particles and drying said fabric. The aqueous solution further comprises deuterium oxide and the concentration of deuterium oxide is about 20% of the overall volume of the aqueous solution. The concentration of the tourmaline particles is about 20% of an overall volume of the aqueous solution. The fabric is fabric is submerged in the aqueous solution for about 72 hours and ionized to about 500 ions per cubic centimeter.

Similarly, fabric could be treated with tourmaline using the same methods described above so that garments could have therapeutic effects. These garments are washable without losing the tourmaline from the fabric.

The use of nano-particles allows more control over the concentration of tourmaline and a more uniform distribution. By targeting specific areas of the body, tourmaline may cause hyperemia in the area and prevent injury, prevent bacterial growth, and expedite healing. Topical application of tourmaline may aid in detoxification, immune system support, anti-microbial effects, analgesia, pain relief, improved focus and self-confidence, and other health benefits.

### DETAILED DESCRIPTION

The above and other objects, features, and advantages of the present disclosure will be more apparent from the following detailed description. Exemplary embodiments are described herein to provide a detailed description of the present disclosure. Variations of these embodiments will be apparent to those of skill in the art.

Moreover, certain terminology is used in the following description for convenience only and is not limiting. The article “a” is intended to include one or more items, and where only one item is intended the term “one” or similar language is used. Additionally, to assist in the description of the present invention, words such as top, bottom, side, upper, lower, front, rear, inner, outer, right and left are used to describe the accompanying figures. The terminology includes the words above specifically mentioned, derivatives thereof, and words of similar import.

The preparation (or gel) and adhesive of the present invention are manufactured with nano-particles of tourmaline, i.e., in powder form. Tourmaline nano-particles having a diameter greater than 100 nanometers can be used to prevent the tourmaline from being absorbed by the skin. Furthermore, such size prevents the tourmaline nano-particles from unwanted dissolution in the solution used to treat fabric and in the binding agent used to manufacture preparations.

Various species of tourmaline can be used including schorl (black), dravite (brown), and elbaite (various colors). The schorl species, characterized by the endmember NaFe2+3A16Si6O18(BO3)3(OH)3OH, is the preferred species due to superior heat generating and heat absorbing qualities. The tourmaline nano-particles are currently sold, for example, under the product name Adan lithia tourmaline by Adan Mining Central (R&D) Labs in Saitama, Japan generally for cosmetics applications.

In one application, the gel of the present invention is manufactured by mixing the tourmaline nano-particles with a binding agent. The binding agent could be any type of solution capable of maintaining a solid property in varied working environments. According to one embodiment, the binding agent is propylene glycol which is an organic compound frequently used in ultrasound gels and electronic cigarettes. Propylene glycol is a viscous, colorless, and odorless liquid that is non-irritating and has low toxicity. Unlike alcohol-based agents, propylene glycol will not cause skin dryness.

Tourmaline powder is added to the binding agent to form a gel that is 20% tourmaline by weight. The resulting tourmaline gel could be used to treat specific parts of the body which requires therapy. The tourmaline gel is also particularly useful for use as a diagnostic gel for ultrasound. The therapeutic gel could be mass produced and sold in containers of any size, shape and material.

In another application, an adhesive is formed with the tourmaline nano-particles. According to an embodiment of a tourmaline adhesive, tourmaline nano-particles are mixed with an acrylic or N-vinylpyrrolidone (“NVP”) polymer to form an adhesive solution that is 20% tourmaline by weight. The adhesive solution is then applied to a breathable and flexible base fabric by spraying to form a self-adhearing bandage or tape. The base fabric is preferably 98% cotton and 2% woven latex. The concentration of tourmaline in the final product is preferably 500 ions per cubic centimeter.

In one embodiment, the base fabric can be applied to garments and reinforced with stitching. In another embodiment, one side of the base fabric is coated with the adhesive solution by spraying, covered with release paper, and conveyed to a rolling device. The bandage or tape is then rolled, coiled, sheared and sealed with a plastic film and packaged for sale. In an alternative embodiment, the base fabric is coated with the adhesive solution on both sides and covered with release paper.

The therapeutic tape or bandage of the present invention allows the fabric to adhere without the use of pins or clips, and without sticking to hair or skin. The base fabric is also constructed in a uniform manner such that it tears evenly as well. The fabric also provides firm compression and maximum support to ligaments, tendons and muscles. The adhesive solution is sufficiently stable for the tape to continue to adhere to the user underwater and is sufficiently stretchable, preferably to more than 150% its original size.

In another embodiment, the adhesive solution is applied directly to garments by spray or other means. The solution can be applied to particular areas of the garment to target specific body parts such as joints. The portions of the garment sprayed with the adhesive solution could then be covered with a non-treated fabric to prevent the garment from adhering to the user's skin.

In another application, the fabric is directly treated with tourmaline. Tourmaline nano-particles are mixed with an aqueous solution such that the concentration of tourmaline is about 20% of total weight of the solution. In one embodiment, the polyacrylic acid derivative Thermacol MP sold by Huntsman in The Woodlands, Tex., is used. A non-treated base fabric, as described above, is submerged in the tourmaline solution and conveyed through one or more heated chambers where the fabric is dried with hot air at approximately 90 degrees Celsius. The resulting therapeutic fabric could be used on its own and could be applied to specific areas of the body where therapy is desired. In the alternative, the resulting therapeutic fabric could be applied with an adhesive, with or without tourmaline, and could be applied as described above to be used as an adhesive tape or bandage.

In another embodiment, the tourmaline nano-particles are mixed with deuterium oxide (“heavy water”) or a combination of heavy water and conventional light water to form an aqueous solution. Again, the concentration of tourmaline is about 20% of the total weight of solution. The base fabric is soaked in the tourmaline solution at room temperature for 72 hours such that the base fabric is ionized and then air dried. Heavy water is denser than conventional light water and is therefore more suitable as a medium for holding tourmaline nano-particles in suspension as dissolution of the tourmaline nano-particles is reduced. Once the base fabric is dried and the heavy water is evaporated, only the tourmaline is left on the finished fabric. This method can reduce manufacturing costs and provide users with a natural product free of chemicals that may cause irritation. The finished fabric has an ideal tourmaline concentration of 500 ions per cubic centimeter.

Similarly, fabric could be treated using the same methods described above to manufacture garments and accessories, i.e., shirts, pants, socks, soft braces, etc. That is, fabric is submerged in the tourmaline solution and conveyed through one or more heated chambers where the fabric is dried with hot air at approximately 90 degrees Celsius. Some or all of the garment or all of the garment could be manufactured with the treated fabric. With the treatment to the fabric, the garment or accessory is washable without losing the tourmaline from the fabric.

It is preferred that the fabric of the present invention is only applied to joint areas of garments. That is, because the treated fabric generates heat, it is ideal to apply the treated fabric to targeted areas rather than the entire garment to optimize comfort for the wearer.

Specific examples of use and application of the preparation and article of the present invention are described below.

The tourmaline athletic tape is manufactured as described in detail above. The therapeutic tape or bandage could also be constructed to attach to clothing. For example, a shirt could be provided with: (i) a tourmaline adhesive in a shape and size that covers the shoulder portion of the shirt while allowing full range of motion; and/or (ii) a tourmaline adhesive strip of a length that covers all or part of the circumference of the waist portion of the shirt. The tourmaline adhesive may be single or double sided and be applied to the inside or the outside of the shirt.

Pants could be constructed with tourmaline attachments as well. For example, pants could be provided with: (i) a tourmaline adhesive strip of a length that covers all or part of the circumference of the waist portion of the pants; (ii) a tourmaline adhesive in a shape and size that covers the hip portion of the pants; and/or (iii) a tourmaline adhesive in a shape and size that covers all or part of the circumference of the knee portion of the pants while allowing full range of motion. The tourmaline adhesive may be single or double sided and applied to the inside or the outside of the pants.

Socks could be constructed with tourmaline attachments. For example, socks could be provided with: (i) a tourmaline adhesive in a shape and size that covers the ankle portion of the socks while allowing full range of motion; and/or (ii) a tourmaline adhesive in a shape and size that covers the toe portion of the socks while allowing full range of motion. The tourmaline adhesive may be single or double sided and applied to the inside or the outside of the shirt.

Elbow, wrist, ankle and knee braces could be constructed with the tourmaline fabric described above. Such braces are constructed in various shapes and sizes that cover specific parts of the body while allowing full range of motion. Optionally, holes could be cut out at or around joints to aid in range of motion.

As described above, tourmaline bandage is manufactured in various shapes and sizes. A sterile pad with or without tourmaline may be attached to the center of a strip of tourmaline adhesive before the release paper is applied.

Therapeutic tape could be used on various parts of the body for various ailments. The therapeutic bandage or tape could be used to treat a number of common injuries including but not limited to carpal tunnel, hamstring strains, wrist pain, runner's knee, neck strain, calf strain, shoulder pain, achilles tendonitis, tennis elbow, plantar fasciitis, golfer's elbow, shin splints, rib pain, ankle sprains, gluteus pain, quad strains and sciatica.

With respect fabrics, the present invention could be used to construct a variety of items. In addition to clothing, domestic items such as sheeting, pillowcases, bed covering and throws could be constructed using methods of the present invention. Also, because of the anti-bacterial effects of the treated fabrics of the present invention, medical wear such as surgical masks, gloves, scrubs and booties could be manufactured using the methods of the present invention.

The concentration of tourmaline in the solutions described above is about 20% total weight and about 500 ions per cubic centimeter in the fabrics described above. These parameters are optimal in generating sufficient heat and ion exchange without discomfort to the user.

As shown above, the present invention provides a cost-effective and convenient means for treating targeted parts of the body with therapeutic and healing effects. The preparations and articles with tourmaline of the present invention solves the problems of the prior art and provides additional advantages.

Although this invention has been described in its exemplary forms with a certain degree of particularity, it is understood that the present disclosure has been made only by way of example and numerous changes in the details of the construction and arrangement of parts may be employed without departing from the spirit and scope of the invention. The scope of the invention will be, therefore, indicated by the claims rather than by the foregoing description. All changes which come within the meaning and range of equivalency of the claims are to be embraced within their scope.

What is claimed is:

1. A therapeutic preparation comprising:
a binding agent; and
tourmaline particles held in suspension in said binding agent.

2. The therapeutic preparation of claim 1, wherein said preparation is selected from a group comprising a gel, a cream, an ointment and a lotion.

3. The therapeutic preparation of claim 1, wherein said binding agent comprises propylene glycol.

4. The therapeutic preparation of claim 1, wherein the tourmaline particles have a diameter greater than 100 nanometers.

5. The therapeutic preparation of claim 1, wherein the tourmaline particles are selected from a group comprising black tourmaline, white tourmaline and gray tourmaline.

6. The therapeutic preparation of claim 1, wherein a concentration of the tourmaline particles is about 20% of an overall volume of the therapeutic preparation.

7. A therapeutic article comprising:
a fabric; and
tourmaline particles attached to said fabric.

8. The therapeutic article of claim 7, wherein the tourmaline particles are ionized into the fabric.

9. The therapeutic article of claim 8, wherein the article is ionized to about 500 ions per cubic centimeter.

10. The therapeutic article of claim 7, further comprising an adhesive applied to the fabric, wherein the tourmaline particles are included in the adhesive.

11. The therapeutic article of claim 10, wherein the adhesive comprises N-Vinylpyrrolidone polymer and wherein a concentration of the tourmaline particles is about 20% of an overall weight of the adhesive.

12. The therapeutic article of claim 10, wherein the adhesive is applied to the fabric by a method selected from a group comprising spraying, soaking, blotting and foaming.

13. The therapeutic tape of claim 7, wherein said fabric is capable of being stretched to at least 150% of its original length.

14. A method of manufacturing a therapeutic article comprising the steps of:
submerging a fabric in an aqueous solution comprising tourmaline particles; and
drying said fabric.

15. The method of claim 14, wherein said aqueous solution further comprises deuterium oxide.

16. The method of claim 15, wherein a concentration of deuterium oxide is about 20% of the overall weight of the aqueous solution.

17. The method of claim 14, wherein a concentration of the tourmaline particles is about 20% of an overall weight of the aqueous solution.

18. The method of claim 14, wherein the fabric is ionized to about 500 ions per cubic centimeter.

19. The method of claim 14, wherein the fabric is submerged in the aqueous solution for about 72 hours.

20. The method of claim 14, further comprising the step of constructing a garment with said therapeutic article.
